(19) United States
(12) Patent Application Publication
(10) Pub. No.: US 2004/0131701 A1
(43) Pub. Date: Jul. 8, 2004

(54) Method to treat infectious diseases and/or to enhance antimicrobial efficacy of drugs

(75) Inventors: Jean Gosselin, Cap Rouge (CA); Pierre Borgeat, Sillery (CA); Louis Flamand, Sainte-Foy (CA); Michel J. Tremblay, Neufchatel (CA)

Correspondence Address: CROWELL & MORING LLP, INTELLECTUAL PROPERTY GROUP, P.O. BOX 14300, WASHINGTON, DC, 20044-4300, US

(73) Assignee: Virocell Inc.

(21) Appl. No.: 10/740,816

(22) Filed: Dec. 22, 2003

Related U.S. Application Data
(63) Continuation of application No. 09/631,637, filed on Aug. 2, 2000.

(51) Int. Cl. A61K 31/555; A61K 33/26

(52) U.S. Cl. 424/646; 514/184

(57) ABSTRACT

The present invention provides a method for the treatment of a viral infection in a patient by administration of a bis-peroxovanadium (bpV) compound, a potent class of phosphotyrosyl phosphatase inhibitors. The method can be utilized for the treatment of patients suffering from infections caused by viruses, such as the human immunodeficiency virus (HIV). The bpV compound may be used in combination with various immunomodulators and/or antiviral agents, in particular, 3TC of which it promotes the phosphorylation into the triphosphate form.

### BACKGROUND OF THE INVENTION

(a) Field of the Invention

The present invention relates generally to a method for the treatment of pathogen-mediated diseases, to a method for enhancing antimicrobial efficacy of antimicrobial agent, and more particularly to a method for the treatment and prevention of diseases caused by viruses, including the human immunodeficiency virus, which comprises the administration of bis-peroxovanadium compounds.

(b) Description of Prior Art

A. Viral Infections in Humans

Viruses are responsible for some of humanity's most devastating pathologies and until recently there existed not one single, truly effective drug for viral infections. Some of the most important and/or common human viral diseases include colds, flu, viral hepatitis, fever-blisters, shingles and acquired immune deficiency syndrome (AIDS). In sharp contrast with what is seen with anti-bacterial drugs, the very few antiviral drugs available are very selective in their activity and each is effective most of the times against only few of the hundreds of viruses that cause human pathologies. Many of the antiviral drugs demonstrate severe shortcomings such as limited efficacy, poor side effects profiles, complicated usage protocols and, more importantly, the frequent emergence of a drug-resistance phenotype.

B. Immune Response to Virus Infections

The immune system can be seen as the controlling factor within the host that will maintain beneficial microbes at harmless levels and prevent infection by dangerous agents such as viruses. The immune system of the host is able to combat a variety of infections from birth. This is accomplished by a system of barriers conferring a generalized or innate immunity. It comprises physical barriers to a to microbial entry, specific phagocytic cells (macrophages), eosinophils, basophils, natural killer cells and various soluble factors, notably the “interferon” complex discovered in the fifties. Interferons are induced upon infection of a variety of cells with viruses. These proteins can trigger the synthesis of several host-cell proteins that contribute to the inhibition of viral replication (2′-5′-oligo-adenylate synthetase), activate a serine/threonine kinase called P1 kinase, increase expression of the MHC-I and TAP transporter proteins, and, finally, activate NK cells. The host also possesses an adaptive specific immunity constituted of humoral and cellular elements, mediated by B cells and antibodies and by T cells, respectively. T cells can recognize foreign antigens as peptides bound to proteins of the major histocompatibility complex class I and II (MHC-I and MHC-II) molecules. Innate immunity is present at all times while adaptive immunity is induced by antigens and gives rise to a long-lasting protection against disease.

C. Evasion of Immune Mechanism by Viruses

Given that immune responses are known to play a key role in the control of virus infections, it is thus not surprising to find that viruses have evolved several mechanisms for evading host immunity. For example, many viruses are capable of great antigenic variation, an event which frequently lead to the development of drug resistance. Some viruses can also suppress immune responses (immunosuppression) by infecting immunocompetent cells, impairing their function and resulting in inhibition of specific immunity, or by mediating the release of soluble factors that may negatively affect other uninfected cells of the immune system. By impairing the immune system viral infections can predispose the patient to other, more serious illnesses of bacterial, fungal, parasitic or even viral origin.

D. HIV-Induced Immunosuppression

Severe immunological abnormalities have been reported to precede the quantitative decline of CD4+ T cell numbers seen in Human Immunodeficiency Virus Type-1 (MIV-1)-infected persons. A decreased stimulation of peripheral blood mononuclear cells (PBMC's) with antibodies specific for CD2 and CD3, with nonspecific mitogens (phytohemagglutinin [PHA] and phorbol 12-myristate 13-acetate [PMA]), and with recall antigens are among abnormalities detected following HIV-1 infection. The exact mechanism(s) responsible for this unresponsiveness (anergy) is still incompletely defined although in vitro studies have shown that signal transduction of the T cell activation pathway was severely impaired. Results from previous studies have demonstrated that defects occurred at the level of intracellular calcium mobilization, membrane depolarization, production of inositol triphosphates, and tyrosine phosphorylation events. It was first proposed, among several possibilities, that the reduced proliferative responses were resulting from the interaction between the external viral envelope glycoprotein gp120 and the CD4 surface glycoprotein because inhibition of T cell receptor (TCR)-dependent proliferative response of PBMC's has been observed following gp120 treatment. The anergic state induced in CD4+ T lymphocytes by gp120 treatment was attributed to inhibition of IL-2 mRNA expression and, consequently, IL-2 secretion since addition of exogenous IL-2 was able to restore proliferative responses. However, very little is known about the intrinsic mechanism(s) implicated in the gpl20-mediated inhibition of IL-2 mRNA production.

E. Limitations of Current Anti-HIV Therapies

The first antiviral agents used to treat individuals infected with HIV were inhibitors directed against the reverse transcriptase, a viral enzyme that is responsible for an early step in the HIV life cycle. Such drugs include AZT (Zidovudine, Retrovir), ddI (Didanosine, Videx), ddC (Zalcitabine, Hivid), d4T (Stavudine, Zerit), 3TC (Epivir, Larivudine), Nevirapine (Viramune), and Delavirdine (Rescriptor). These compounds have significantly helped the treatment of HIV-1-infected persons, but, unfortunately, their beneficial effects are markedly limited by their inherent significant toxicity and the rapid apparition of resistant viral strains. The development of a new class of drugs, the protease inhibitors, has improved the efficacy of the anti-HIV therapy. The virus-encoded protease is an enzyme that cleaves some HIV proteins at several sites to complete formation of infectious viral particles. Although treatment with protease inhibitors alone resulted in driving virus levels below the limits of detection in peripheral blood, many patients have suffered relapses concomitant with the development of HIV resistant to protease inhibitors. The various drawbacks associated with monotherapy have led to new antiretroviral therapies combining inhibitors of HIV-1 reverse transcriptase and protease, a mixture of antiviral drugs better known as highly active antiretroviral therapy (HAART). The advent of HAART for the care of people with HIV-1 infection has led to a dramatic reduction in viral load and, consequently, to a significant decline in the incidence of AIDS and in mortality from this retroviral infection. Unfortunately, several groups have reported in late 1997 that infectious progeny viruses could still be isolated even from patients receiving HAART for considerable periods of time (up to 3 years) and in whom plasma viral load was below the detection limit of the current most sensitive assays, These observations have led to the concept that persistent cellular reservoirs existed in HIV-1-infected individuals into which the virus remained latent. Moreover, increasing numbers of treatment failures resulting from toxicity, drug-resistant mutants and poor compliance of patients to drug regimen are emerging with long-term therapy. Another limitation of the treatment of HIV-1-infected persons with the available regimens of HAART resides in the partial regeneration of non-HIV-1-specific immune responses and a weak restoration of HIV-1-specific responses. Therefore, for HAART to be more effective in the treatment of HIV-1-infected individuals, immunomodulators are now seriously considered as important additions to this pharmacologic arsenal to achieve long-term control and, hopefully, to complete eradication of the virus. Several strong T-cell activators are thus potential candidates for increasing the immune response in HIV-1-infected persons.

F. Protein Tyrosine Phosphatases and Cellular Activation

Phosphorylation of tyrosine residues of intracellular proteins is regulating almost every aspect of cellular function including cell growth, proliferation, differentiation and T cell activation. The process of protein tyrosine phosphorylation is tightly controlled by the dynamic balance between protein tyrosine kinase and protein tyrosine phosphatase activities. Therefore, it is not surprising to find that the protein tyrosine phosphatases (PTPs), enzymes responsible for the dephosphorylation of proteins on their tyrosine residues, are also very important modulators of T cell activation cascade. PTPs are thus generally presented as inhibitors of T cell activation and this has been more clearly indicated by studies of the protein tyrosine phosphatase SHP-1. PTSs have also been shown to be important players in the cascade leading to the activation of transcription factors in T cells. Some investigators have indeed used the pervanadate PTP inhibitor to activate NF-κB in T cells. Although the studies on the mechanism of activation have reported some confusing data, these latter results have exposed the importance of PTPs in the control of NF-κB activation. Imbert and co-workers have further demonstrated that AP-1 could also be activated by pervanadate in T cells, while another group has shown the induction of STAT proteins via the activation of the tyrosine kinase Jak1 in a different experimental setting. Recently, it was shown that treatment with a new set of PTP inhibitors, the bis-peroxovanadiums (bpV) compounds, resulted in the activation of the HIV-1 LTR in T cells partly through the activation of NF-κB. However, this work had also demonstrated the implication of an NF-κB-independent pathway which was induced by these same inhibitors. Since tyrosine phosphorylation plays such a cardinal role in most cascades leading to T-cell activation, PTP inhibitors are to be considered as potential agents to compensate for T-cell anergy observed in HIV-1-infected individuals. This idea is supported by the observation that pervanadate, a potent PTP inhibitor, has been demonstrated to lead to T-cell activation.

G. Bis-peroxovanadium Compounds, a Novel Series of Highly Potent Protein Tyrosine Phosphatase Inhibitors

The role played by protein tyrosine phosphatases (PTPs) in the molecular physiology of haematopoietic cells has been investigated primarily through the use of specific inhibitors, Vanadate is a well-documented inhibitor of PTPs. Previous studies have indicated that the combination of vanadate (V⁵⁺) and hydrogen peroxide (H₂O₂) generates the compound pervanadate, the efficacy of which has been demonstrated to be far superior than that of vanadate. This synergy between vanadate and hydrogen peroxide was postulated to result from the formation of aqueous peroxovanadates, created by the peroxide ion forming a complex with vanadium. However, the very poor stability of aqueous peroxovanadates and the multitude of species in complex equilibrium led to the discovery of new, stable and structurally defined bis-peroxovanadium (bpV) complexes that can be easily distinguished using ⁵¹V nuclear magnetic resonance. In a typical bpV compound, the vanadium ion occupies the central position of the pentagonal biptramid, with two peroxo groups in the pentagonal plane. The single oxo group is positioned perpendicular to the pentagonal plane (axial). The remaining positions are filled with an ancillary ligand located in the inner coordination sphere of vanadate. The presence of the ancillary ligand confers greater kinetic stability upon bpV complexes compared with vanadate or aqueous peroxovanadates.

It would be highly desirable to be provided with a method for the treatment of pathogen-mediated diseases, to a method for enhancing antimicrobial efficacy of antimicrobial agent, and more particularly to a method for the treatment and prevention of diseases caused by viruses, including the human immunodeficiency virus, which comprises the administration of bis-peroxovanadium compounds.

SUMMARY OF THE INVENTION

One aim of the present invention is to provide a method for the treatment of pathogen-mediated diseases, to a method for enhancing antimicrobial efficacy of antimicrobial agent, and more particularly to a method for the treatment and prevention of diseases caused by viruses, including the human immunodeficiency virus, which comprises the administration of bis-peroxovanadium compounds.

In accordance with the present invention there is provided a method for the treatment of an infection in a patient, which comprises administering to said patient a therapeutically effective amount of a bis-peroxovanadium (bpV) compound.

The bpV compound may be a phosphotyrosyl phosphatase inhibitor and/or may comprises an oxo ligand, two peroxo anions, and an ancillary ligand located in an inner coordination sphere of vanadate. The infection may be caused by a virus.

The patient is preferably a mammal which may be selected from the group consisting of human, ovine, bovine, equine, caprine, porcine, feline and canine.

The virus may be a human virus selected from the group consisting of DNA viruses, RNA viruses and Retroviridae, preferably the virus is a human immunodeficiency virus.

The bpV compound may be administered intravenously, subcutaneously, intradermally, transdermally, intraperitoneally, orally or topically.

The bpV compound may be administered with a patch or an implant.

The bpV compound may be administered by inhalation, such as with an aerosol spray or in a powder form.

The bpV compound may be in association with a liposomal composition suitable for administration.

The bpV compound may be in a tablet form.

The bpV compound may be administered in combination with an antiviral agent, which include, without imitation, nucleoside analogues, protease and neuraminidase inhibitors, interferon α, and non nucleoside analogues, such as non nucleoside reverse transcriptase inhibitors (NNRTI), chemokines and chemokines antagonists.

The antiviral agent is preferably AZT and/or 3TC.

The bpV compound may be administered in combination with one or more immunomodulator(s) which includes, without limitation, leukotrienes, chemokines, cytokines, growth factors and interferons. Preferably, such immunomodulators include, without limitation, leukotriene B4, IL-2, G-CSF, GM-CSF, interferon β and γ

In accordance with another embodiment of the present invention, there is provided a method for the enhancement of antimicrobial efficacy of antimicrobial agents, which comprises administering to a patient undergoing an antimicrobial therapy, a therapeutically effective amount of a bis-peroxovanadium (bpV) compound.

Preferably, the bpV compound is a phosphotyrosyl phosphatase inhibitor. More preferably, the bpV compound comprises an oxo ligand, two peroxo anions, and an ancillary ligand located in an inner coordination sphere of vanadate.

The antimicrobial agent is selected from the group consisting of nucleoside analogues, protease and neuraminidase inhibitors, interferon α, and non nucleoside analogues, such as non nucleoside reverse transcriptase inhibitors (NNRTI), chemokines and chemokines antagonists.

In accordance with another embodiment of the present invention, there is provided a pharmaceutical composition for the treatment of an infection in a patient, which comprises an therapeutically effective amount of a bis-peroxovanadium (bpV) compound in association with a pharmaceutically acceptable carrier.

### BRIEF DESCRIPTION OF THE DRAWINGS

FIGS. 1A and 11 illustrates bar charts indicating that bpV[pic] and bpv[phen] compounds markedly diminish HIV-1 replication in Sup-T1 cells at subcytotoxic concentrations;

FIGS. 2A and 2B show that bpv[pic] and bpV[phen] compounds inhibit infection of PM1 cells with both T- and macrophage-tropic isolates of HIV-1;

FIG. 3 illustrates that pretreatment of primary human monocyte-derived macrophages (MDM) with bpV[pic] and bpV[phen] molecules decreases the process of infection with HIV-1;

FIG. 4 depicts toxicity of bpV[pic) and bpV[phen] compounds in primary human MDM;

FIGS. 5A and 5B illustrate bar charts showing an additive antiviral effect between bpV compounds and two widely used nucleoside reverse transcriptase inhibitors, namely AZT and 3TC;

FIG. 6A depicts a bar chart indicating an additive anti-HIV-1 effect between bpV[pic] and 3TC when primary human MDM are used as targets;

FIG. 7 illustrates a bar chart showing an additive anti-HIV-1 effect between increasing concentrations of bpV[pic] molecule and 3TC; and

FIG. 8 shows a bar chart indicating that treatment of human primary peripheral blood mononuclear cells with bpV[pic] leads to an increase of the ratio of the active antiviral triphosphate form of 3TC over the diphosphate form.

### DETAILED DESCRIPTION OF THE INVENTION

In accordance with a preferred embodiment, the present invention relates to the use of bpV compounds, a new class of potent phosphotyrosyl phosphatase inhibitors, in the treatment of humans suffering from a pathogen-mediated diseases.

The present invention comprises a class of biologically compounds which are acting as potent protein tyrosine phosphatase inhibitors which are useful in treating various pathological conditions in humans such as diseases caused by viruses.

bpV compounds are made of an oxo ligand, two peroxo anions, and an ancillary ligand located in the inner coordination sphere of vanadate.

Ancillary ligands located in the inner coordination sphere of the vanadate atom include bipyridine [bipy]; picolinic acid (pyridine-2-carboxylic acid) anion [pic]; 5-hydroxypyridine-2-carboxylic acid anion (HO-pic]; 1,10-phenanthroline [phen]; 4,7-dimethyl-1,10-phenanthroline [Me2phen]; 3)4,7,8-tetamethyl-1,10-phenanthroline [Me4phen]; oxalic acid dianion [ox].

Formulas and abbreviations of a number of structurally defined bpV compounds are listed herein to illustrate the invention rather than to limit it: K[VO(O2)2bipy].5H2O, bpV [bipy]; K2[VO(O2)2pic].H2O, bpV [pic]; K2[VO(O2)2(EOpic)].H2O, bpV [Hopic]; K[VO(O2)2phen]0.3H2O, bpV [phen]; K[VO(O2)2(4,7-Ne2phen)]; bpV [Me2phen]; K[VO(O2)2(Me4phen)], bpV [Me4phen]; and K3[VO(O2)2OX].2H2O, bpV[ox].

Treatment with bpV compounds represent a new therapeutic avenue to treat humans infected with viruses.

In relation to Examples 1 to 8 given hereinafter, the materials used and the analyses and assays carried out were as follows:

Cells

Target cells used in the present invention include human CD4-expressing T lymphoid Sup-T1, PM1, and Jurkat cells. Moreover, primary human monocyte-derived macrophages (MDM) and peripheral blood mononuclear cells (PBMC′) were also used in the current work, MDM were obtained using a standard technique. In brief, the mononuclear cell fraction was isolated by Ficoll-Hypaque centrifugation. Peripheral blood mononuclear cells were suspended in seeding medium (RPMI 1640+20% fetal calf serum+10% human serum type AB) in tissue culture 48-well plates (3×10⁶ cells/ml and 500 μl per well). Five days after the initiation of the cultures, nonadherent cells were removed by rinsing the cultures three times with phosphate buffered saline. Next, such adherent cells were maintained in RPMI 1640 medium supplemented with 20% fetal calf serum.

Preparation of bpV Compounds

bpV molecules were prepared as described previously (Posner et al., J. Biol. Chem. 269:4596-4604, 1994). Briefly, V₂O₅ was dissolved in an aqueous KOH solution and then mixed with 30% H₂O₂ and the respective ancillary ligand in addition to the ethanol for optimal precipitation. Characterization of the bpV molecules were carried out by infrared 1H-NM and Vanadium-51 (⁵¹V) NMR spectroscopy. Stock solutions of bpV molecules (1 mM in phosphate buffered saline pH 7.4) were kept at −85° C. until use.

Virus Preparations

Fully infectious viral entities were generated by calcium phosphate transfection of 293T with pNL4-3 vector (T-tropic virions) as described below. Recombinant luciferase-encoding virus particles pseudotyped with the appropriate Env proteins have been used in our series of investigations. Such a system provides a highly sensitive and reproducible assay to monitor single-cycle viral infection events. This test is based on the molecular construct pNL4-3-Luc-E-R+, a vector that carries the gene for firefly luciferase inserted into the nef gene of the pNL4-3 provirus and contains also a frameshift at the 5′ end of env (nt 5950) that prevents expression of the envelope glycoproteins. Progeny viruses were generated by cotransfecting 293T cells with pNL4-3-Luc-E-R+ and a plasmid DNA encoding for the appropriate envelope glycoproteins (T or macrophage-tropic). Briefly, a typical transfection experiment was performed as follow. In brief, 293T cells were plated 24 h before transfection at a concentration of 5×10⁵ cells per 3 ml of DMEM into each well of 6-well plates. All solutions were brought to room temperature before use. Immediately before transfection, DNA was added to 25 μl of 2.5M CaCl₂ and the volume was completed to 250 μl with distilled water. This solution was then added drop by drop to 250 μl of 2×HBS solution (280 mM NaCl, 50 mM HEPES, 1.5 mM Na₂HPO₄, pH 7.05) and the resulting mixture was stored at room temperature for 5 min. This DNA-HBS mixture was finally added drop by drop to plated 293T cells before incubation at 37° C. under a 5% CO₂atmosphere. At 16 h after transfection, cells were washed twice with 3 ml of PBS and were incubated for an additional 24 h with 3 ml of DMEM supplemented with 10% FBS. Virion-containing supernatants were filtered through a 0.45-μm cellulose acetate membrane (Millipore, Mass.), aliquoted in 200 μl fractions, and were finally frozen at −85° C. until needed. Virus stocks were normalized for virion content using a commercial assay for the viral major core protein p24 (Organon Teknika, Durham, N.C.).

Measurement of Virus-Encoded Luciferase Activity

In brief, 100 μl of cell-free supernatant were discarded from each well and 25 μl of cell culture lysis buffer 5×(125 mM Tris phosphate [pH 7.8], 10 mM DTT, 5% Triton X-100, and 50% glycerol) were added to the wells before incubation at room temperature for 30 min. An aliquot of cell lysate (20 μl) was mixed with 100 μl luciferase assay buffer (20 mM tricine, 1.07 mM (MgCO₃)₄. Mg(OH)₂.5H₂O, 2.67 mM MgSO₄, 0.1 mM EDTA, 270 μM coenzyme A, 470 μM luciferin, 530 μM ATP, and 33.3 mM DTT). Luciferase activity was monitored using a microplate luminometer (MLX; Dynex Technologies, Chantilly, Va.).

HPLC Analysis of Phosphorylated Forms of 3TC

Analysis of the cellular content in 3TC and its phosphorylated metabolites was performed by HPLC as follows. In preparations for HPLC analysis, 1 ml of a mixture of acetonitrile/water (5/95, vol/vol) and 0.5 ml water were added to each sample. The samples were vortexed and centrifuged at 1000×g for 10 min at room temperature to remove the precipitated material. The supernatants were collected and used without further treatment for HPLC analysis. Two (2) ml of each sample (total volume of ˜2.5 ml) were injected onto a Beckman Ultrasphere ODS column (4.6 mm×150 mm) protected with a Guard-Pak C18 (Waters Millipore) pre-column. 3TC and its metabolites were eluted using a linear gradient from 100% solvent A to 100% solvent B in 25 minutes at a flow rate of 1.2 ml/minute (Solvent A: 5% acetonitrile, 70% water, 25% buffer [40 mM H3PO4, 8 mM tetrabutylammonium hydroxide, adjusted to pH 6.75 with concentrated ammonium hydroxide]; Solvent B: 40% acetonitrile, 35% water, 25% buffer). The elution of metabolites was monitored by counting radioactivity using a Flow Scintillation Analyser 500TR Series (Packard); the HPLC column effluent (1.2 ml/min) was mixed with a liquid scintillation cocktail (3 ml/min) (Ultima Flow M, Packard).

Dose Ranges

The therapeutically effective amount of the inhibitor of the present invention to be administered will vary with the particular inhibitor used, the type or mode of administration, the concurrent use of other active compounds, host age and size, type, response of individual patients, and the like. In the case of bpV compounds, it will be administered in sufficient doses to obtain an effective peak or steady-state concentration of about 100 nM to 25 μM, usually about 10 μM in plasma as suggested by the concentrations of bpV compounds tested and found to be effective in in vitro experiments. An effective dose amount of the bpV compounds is thus to be determined by the clinician after a consideration of all the above-mentioned criteria. The dosage amount of agent necessary to obtain the desired concentrations in blood can be determined by pharmacokinetic studies.

Pharmaceutical Compositions

Any suitable type or mode of administration may be employed for providing a mammal, especially a human with an effective dosage of a bpV compound of the present invention. For example, oral, parenteral and topical may be employed. Dosage forms include tablets, capsules, powders, solutions, dispersions, suspensions, creams, ointments and aerosols.

The pharmaceutical compositions of the present invention comprise a bpV compound as a phosphotyrosyl phosphatase inhibitor and as the active ingredient, and a pharmaceutically acceptable carrier and optionally other therapeutic ingredients.

It should be recognized that the bpV compounds can be used in a variety of ways in vivo. It can be formulated into pharmaceutical compositions according to any known methods of preparing pharmaceutically useful compositions. In this manner, the bpV compounds are combined in admixture with a pharmaceutically acceptable carrier vehicle. Suitable vehicles and their formulation, including human proteins, such as human serum albumin, are described for instance in Remington's Pharmaceutical Sciences (16th ed. Osol, A., ed., Mack, Easton, Pa. [1980]). In order to form a pharmaceutically acceptable composition suitable for effective administration, such compositions will contain a therapeutically effective amount of the bpV compound, together with a suitable amount of carrier vehicle. The therapeutically effective concentration of the bpV compounds can be determined by in vivo pharmacological studies.

The bpV compound can be formulated as a sterile pharmaceutical composition for therapeutic use which is suitable for intravenous or intraarterial administration. The product may be in a solvent-free form and ready to be reconstituted for use by the addition of a suitable carrier or diluent, or alternatively, it may be in the form of solution which may be aqueous or organic.

For reconstitution of a solvent-free product in accordance with the present invention, one may employ a sterile diluent, which may contain materials generally recognized for approximating physiological conditions. In this manner, the sterile diluent may contain salts and/or, buffering agents to achieve a physiologically acceptable tonicity and pH, such as sodium chloride, phosphate and/or other substances which are physiologically acceptable and/or safe for use.

When used as an aqueous solution, the pharmaceutical composition will for tie most part contain many of the same substances described above for the reconstitution of a solvent-free product. When used in solution in an organic solvent, a small volume of the solution containing the bpV compound will be diluted with an aqueous solution that will contain many of the same substances described above for the reconstitution of a solvent-free product. The pharmaceutical composition, for the most part, will thus contain many of the same substances described above for the reconstitution of a solvent-free product.

The bpV compound useful in the methods of the present invention may be employed in such forms as, for example, sterile solutions for injection or encapsulated (for instance in liposomes) or embedded (for example in suppositories) for slower long-lasting release.

The bpV compound may be used in combination with other agents including, but not limited to, anti-viral agents or other immunomodulator.

Where the subject bpV compound is to be administered to a host as an inhibitor of phosphotyrosyl phosphatase, the bpV compound may be administered, for example, intraarterially, intravenously, intraperitoneally, subcutaneously, intramuscularly, by injection, by suppository, by inhalation, or the like.

The mode of administration by injection includes continuous infusion as well as single or multiple boluses. Useful administration type or mode also includes the use of implantable internal pumps for continuous infusion into a blood vessel or at different sites such as the peritoneal cavity or subcutaneously. Such techniques are disclosed in Cecil's Text Book of Medicine (chapter 164, 19th Edition, 1992) for the treatment of hepatic cancers. Transdermal administration by means of a patch containing the bpV compound of the present invention may also be a useful administration mode.

Additional pharmaceutical methods may be employed to control the duration of action. For example, controlled release preparations may be achieved through the use of macromolecules to complex or absorb the bpV compound. The controlled delivery may be achieved by selecting appropriate macromolecules (for example, polyesters, polyamino acids, polyvinyl pyrrolidone, ethylene-vinyl acetate, methyl cellulose, carboxymethyl cellulose, protamine sulfate or serum albumin, the appropriate concentration of macromolecules, as well as the methods of incorporation). In this manner, release of the bpV compound can be controlled.

Another possible method useful in controlling the duration of action by controlled release preparations is the incorporation of the bpV compound into particles of a polymeric material such as polyesters, polyamino acids, hydrogels, poly(lactic acid), or ethylene-vinyl acetate copolymers.

Instead of incorporating the subject bpV compound into polymeric particles, it is also possible to entrap them in microcapsules prepared, for instance, by coacervation techniques or by interfacial polymerization (for example, hydroxymethyl cellulose or gelatin microcapsules and polymethyl methacrylate microcapsules, respectively), in colloidal drug delivery systems (for example, liposomes, albumin microspheres, microemulsions, nanoparticles and nanocapsules) or in macroemulsions. Such techniques are disclosed in Remnington's Pharmaceutical Sciences (16th ed. Osol, A., ed., Mace Easton, Pa. [1980]).

The compositions include compositions suitable for oral or parenteral administration. Conveniently they are presented in unit dosage form and prepared by any of the methods well-known in the art of pharmacy.

In practical use, the bpV compound can be combined as the active ingredient in intimate admixture with a pharmaceutical carrier according to conventional pharmaceutical compounding techniques. The carrier may take a wide variety of forms depending on the form of preparation desired for administration. In preparing thc compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions; elixirs and solutions; or carriers such as starches, sugars, microcrystalline cellulose, diluents, granulating agents, lubricants, binders, disintegrating agents and the like in the case of oral solid preparations such as, for example, powders, capsules and tablets. If desired, tablets may be coated by standard aqueous or nonaqueous techniques.

Pharmaceutical compositions of the present invention suitable for oral administration may be presented as discrete units such as capsules, cachets or tablets each containing a predetermined amount of the bpV compound, as a powder or granules or as a solution or suspension in an aqueous liquid, a non-aqueous liquid, an oil-in-water emulsion or a water-in-oil emulsion. Such compositions may be prepared by any of the methods of pharmacy such methods including the step of bringing the bpV compound into association with the carrier which includes one or more necessary ingredients. In general, the compositions are prepared by uniformly and intimately admixing the bpV compound with liquid carriers or finely divided solid carriers or both, and then, if necessary, shaping the product into the desired presentation. For example, a tablet may be prepared by compression of molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

It will be understood that the bpV compound is to be administered in pharmacologically or physiologically acceptable amounts, by which is to be understood amounts not harmful to the patient, or amounts where any harmful side effects in individual patients are outweighed by the benefits. Similarly, the bpV compound is to be administered in a therapeutically effective amount, which is to be understood is an amount meeting the intended therapeutic objectives, and providing the benefits available from administration of bpV compound.

The present invention will be more readily understood by referring to the following examples which are given to illustrate the invention rather than to limit its scope.

### EXAMPLE 1

### Replication of HIV-1 in Sup-T1 Cells is Diminished by Subcytotoxic Concentrations of bpv[pic] and bpv[pben] Compounds

Sup-T1 cells were seeded at a density of 10⁵ cells per well (100 μl) in 96-well flat-bottom, plates. Cells were either left untreated (control) or were pretreated for 5 min at 37° C. with the two different bpV molecules (bpV [pic] and bpV[phen]) at the indicated concentrations in a final volume of 200 μl. Next, both untreated and bpV-treated Sup-T1 cells were inoculated with the fully infectious T-tropic strain HIV-1_{NL4-3} (10 ng of p24). Culture medium was replaced twice a week and was supplemented with the appropriate final concentration of bpV molecules. Cells were incubated for 10 days and kinetics of viral infection was assessed by measuring in cell-free culture supernatants the major core viral p24 protein with the use of a commercial enzymatic assay (Organon Teknika). Putative toxicity of bpV molecules was assessed by adding the tetrazolium salt MTT to Sup-T1 cells that were cultured for 10 days under the constant pressure of the indicated concentrations of bpV[pic] and bpV[phen] molecules.

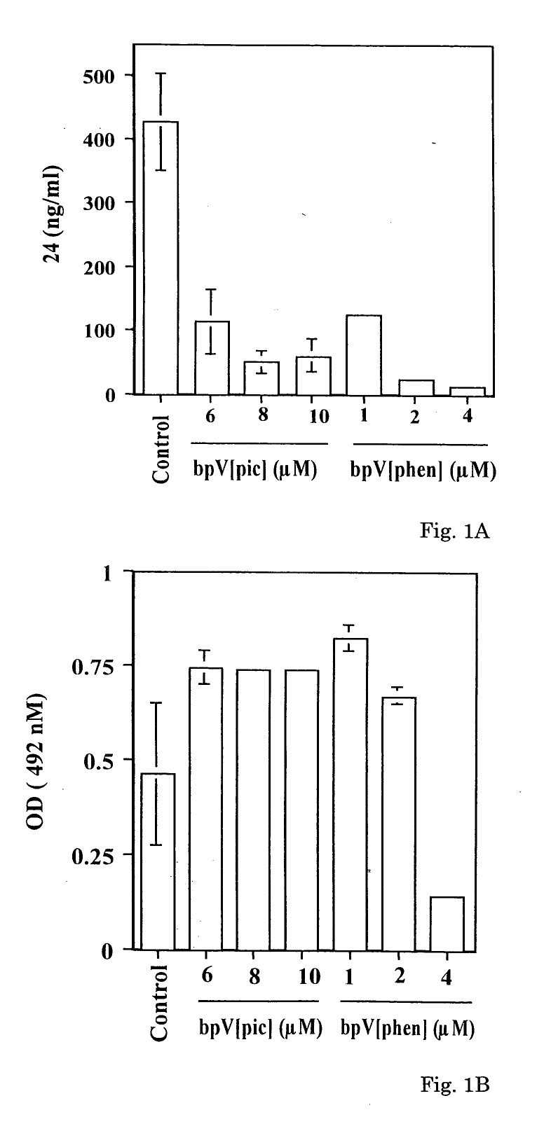

Results from FIG. 1A indicate that treatment of human T lymphoid Sup-T1 cells with bpV[pic] and bpV[phen] leads to a dramatic decrease of HIV-1 production. This antiviral effect was seen at all three tested concentrations. Values shown represent the mean of three different measured samples±S.D. This is representative of two independent experiments.

Data from FIG. 1B demonstrate that bpv[pic] at all three tested concentrations has no toxic effect on Sup-T1 cells despite a 10 days treatment. However, a detectable toxicity was observed with the maximal concentration of bpV[phen] tested (4 μM).

### EXAMPLE 2

### Infection of PM1 Cells by T- and macrophage-tropic Strains of HIV-1 is Decreased by bpV[pic] and bpv[phen] Compounds

Our next series of investigations was carried out using PM1, a human CD4-, CXCR4-, and CCR5-positive T lymphoid cell line known to be susceptible to infection with both T- and macrophage-tropic strains of HIV-1. PM1 cells were seeded at a density of 3×10⁴ cells per well (100 μl) in 96-well flat-bottom plates. Cells were either left untreated (control) or were pretreated for 5 min at 37° C. with the two different bpV molecules (bpV[pic] and bpV[phen]) at the indicated concentrations in a final volume of 200 μl. Next, both untreated and bpV-treated PM1 cells were infected with luciferase reporter viruses bearing T- (panel A) or macrophage-tropic (panel B) envelope proteins (10 ng of p24). Next, PM1 cells were kept incubated at 37° C. for 72 h and were lysed before monitoring luciferase activity with a mircroplate luminometer. Results shown are the mean±SD for triplicate samples and are representative of two independent experiments.

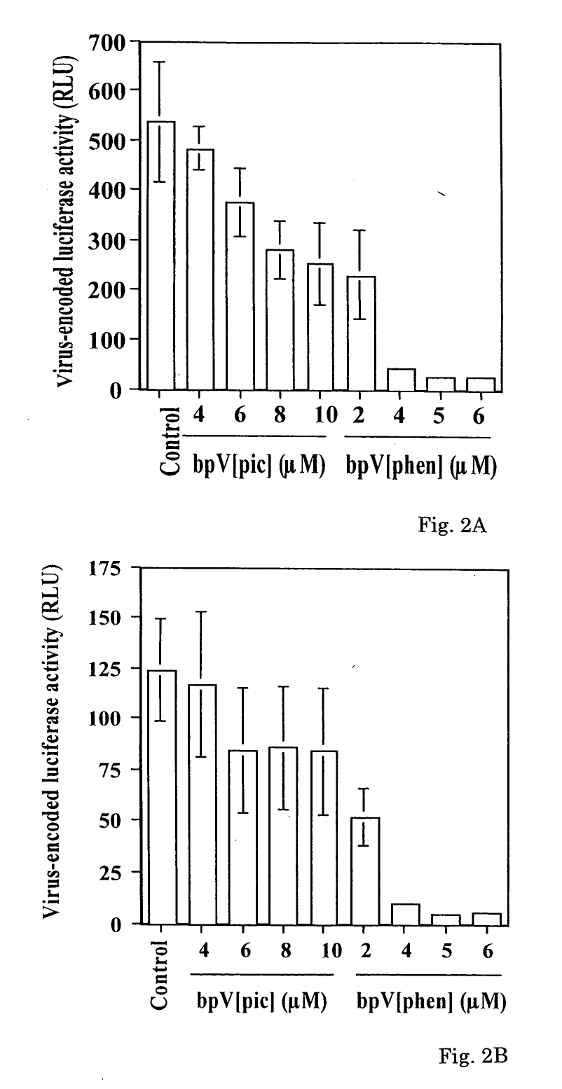

Results from FIGS. 2A and 213 indicate that pretreatment of PM1 cells with bpV[pic] and bpV[phen] compounds results in a dose-dependent inhibition of infection with T- and macrophage-tropic recombinant luciferase-encoding HIV-1 particles.

### EXAMPLE 3

### Infection of Primary Human MDM by Macrophage-Tropic HIV-1 is Decreased by bpV[pic] and bpV[phen] Molecules

Primary human monocyte-derived macrophages (MDM), which were obtained by plastic adherence for 5 days in 48-well plates, were first pretreated or not for different time periods (5, 15, 30, 60, and 120 min) at 37° C. either with 10 μM bpV[pic] or 5 μM bpV[phen]. MDM were subsequently infected with recombinant luciferase-encoding virions (NL4-3 backbone) pseudotyped with macrophage-tropic ADA envelope (10 ng of p24). Infection was allowed to proceed for 48 h and MDM were lysed before monitoring luciferase activity with a microplate luminometer (MLX; Dynex Technologies, Chantilly, Va.). Results shown are the mean±SD for triplicate samples and are representative of two independent experiments.

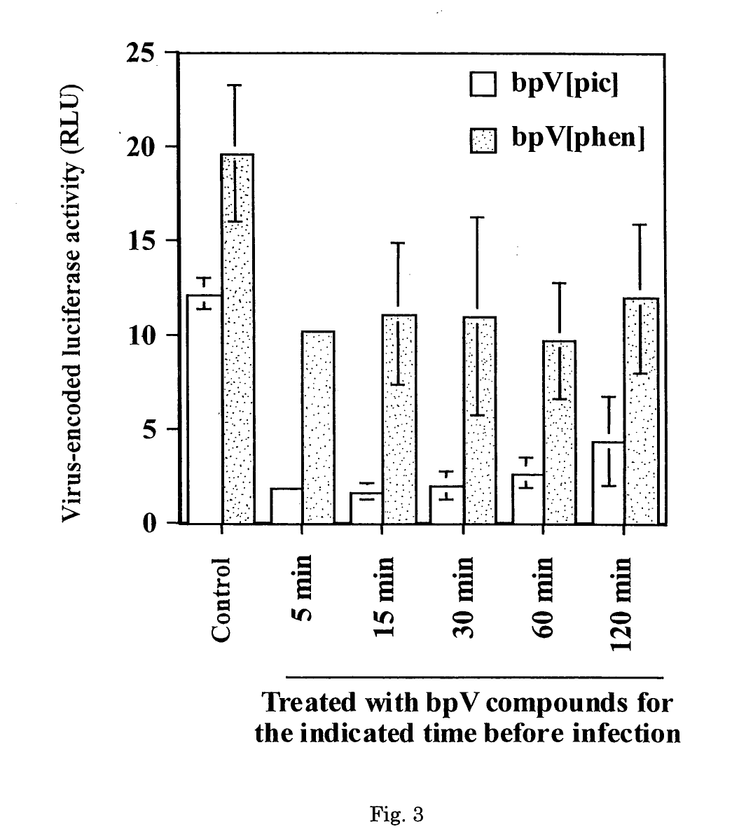

FIG. 3 shows that the anti-HIV-1 efficacy of bpV[pic] and bpV[phen] on MDM is maintained over several pretreatment periods ranging from 5 to over 120 minutes.

### EXAMPLE 4

### Toxicity of bpV[pic] and bpV[phen] Compounds in Primary Human MDM

Putative toxicity of bpV[pic] and bpV[phen] molecules was next assessed by adding the tetrazolium salt MTT to primary human MDM that were cultured for 48 h under the constant pressure of the indicated concentrations of bpV compounds.

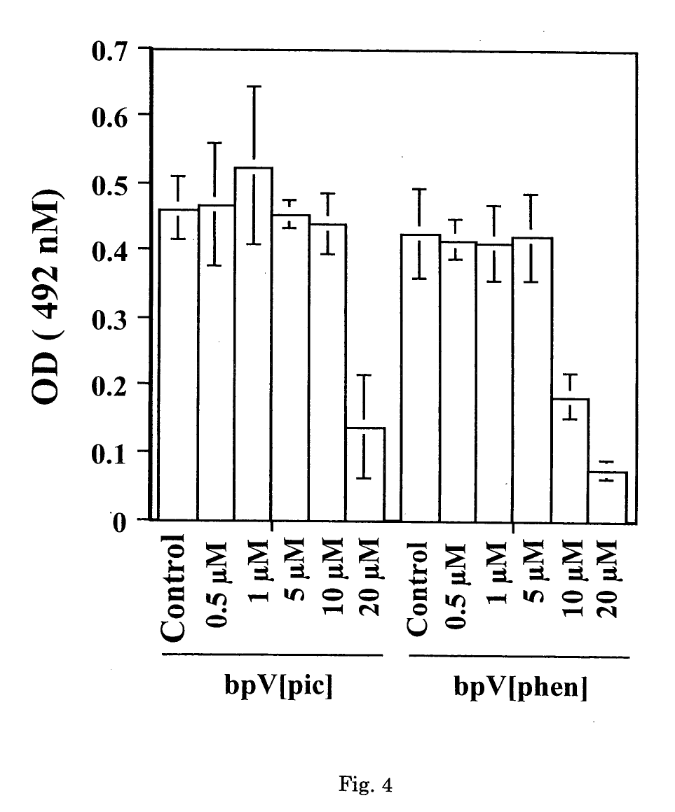

Results from FIG. 4 demonstrate that bpV[pic] is toxic for MDM at 20 μM, whereas bpV[phen] has detectable toxic effect at 10 and 20 μM.

### EXAMPLE 5

### Additive Anti-HIV-1 Effect Between bpV Compounds and Two Widely Used Nucleoside Reverse Transcriptase Inhibitors, Namely AZT and 3TC

We attempted to define any putative interaction (additive, synergistic or antagonistic) between bpV compounds and currently approved anti-HIV-1 agents (AZT/Zidovudine, 3TC/Epivir/Lamivudine). First, human T lymphoid Jurkat cells (1×10⁵) were pretreated for 5 min at 37° C. with increasing concentrations of either bpV[pic] (2.5, 5, and 10 μM) (panel A) or bpV[phen] (1.25, 2.5, and 5 μM) (panel B). Jurkat cells were also pretreated with the indicated concentrations of bpV[pic] or bpV[phen] in combination with either 0.05 μM AZT or 0.05 μM 3TC. Jurkat cells were then infected with recombinant luciferase-encoding virions (NL4-3 backbone) pseudotyped with T-tropic HXB-2D envelope (10 ng of p24). Infection was allowed to proceed for 72 h and Jurkat cells were lysed before monitoring luciferase activity with a microplate luminometer (MLX; Dynex Technologies, Chantilly, Va.). Results shown are the mean±SD for triplicate samples and are representative of two independent experiments.

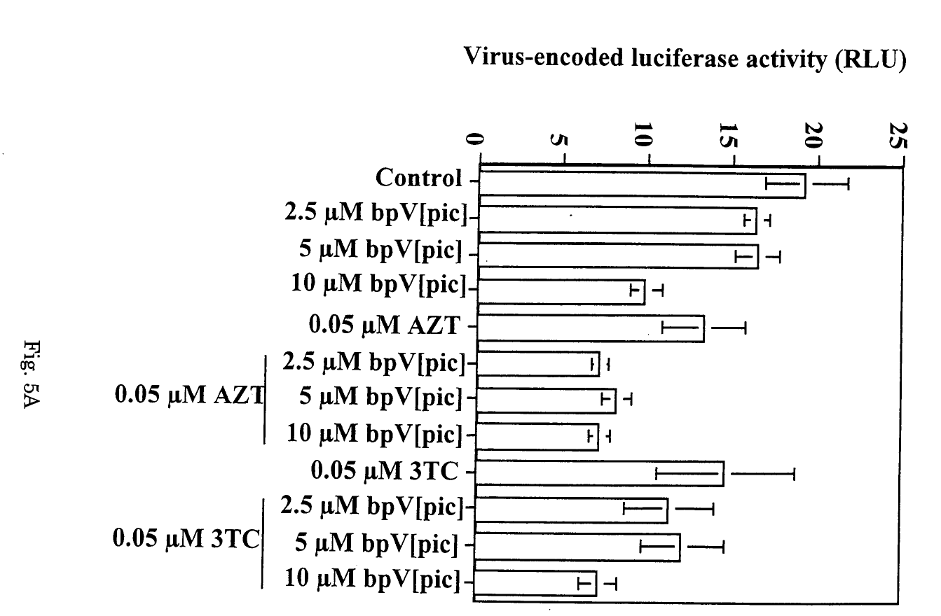

As illustrated in FIG. 5A, bpV[pic] inhibits HIV-1 infection of Jurkat cells. The process of virus infection was also decreased when using the two antiviral agents AZT and 3TC. Interestingly, a greater antiviral effect was reached when bpV[pic] was used in combination with AZT or 3TC. A similar observation was made when either AZT or 3TC was combined with bpV[phen] (FIG. 5B). These findings suggest that an additive antiviral effect is obtained when bpV compounds are used in conjunction with nucleoside reverse transcriptase inhibitors such as AZT and 3TC.

### EXAMPLE 6

### Additive Anti-HIV-1 Effect Between bpV[pic] and 3TC When Using Primary Human MDM as Targets

Primary human monocyte-derived macrophages (MDM), which were obtained by plastic adherence for 5 days in 48-well plates, were first pretreated or not for 15 min at 37° C. with bpV[pic] (10 μM), 3TC (0.035 μM), and bpV[pic]/3TC combination. MDM were subsequently infected with recombinant luciferase-encoding virions (NL4-3 backbone) pseudotyped with macrophage-tropic ADA envelope (10 ng of p24). Infection was allowed to proceed for 48 h and MDM were lysed before monitoring luciferase activity with a microplate luminometer (MLX; Dynex Technologies, Chantilly, Va.). Results shown are the mean+SD for triplicate samples and are representative of two independent experiments.

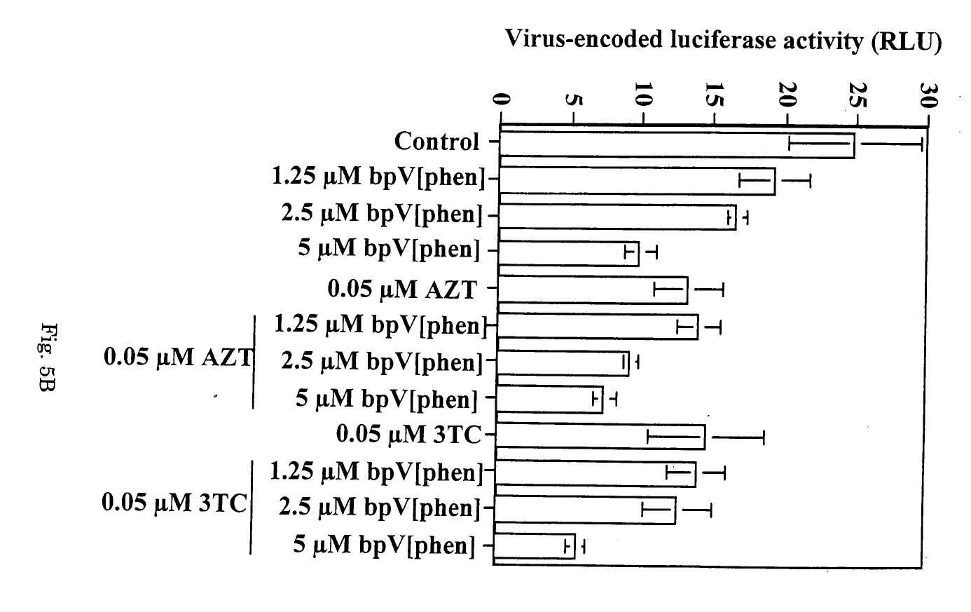

Data from FIG. 6 confirm our previous findings that bpV[pic] alone inhibits HIV-1 infection of primary human MDM. As expected, 3TC was also able to diminish the process of virus infection in such target cells. More importantly, an additive antiviral effect was seen in primary human MDM when both bpV[pic] and 3TC were used in combination.

### EXAMPLE 7

### Additive Anti-HIV-1 Effect Between 3TC and Increasing Concentrations of bpV[pic]

Primary human monocyte-derived macrophages (MDM), which were obtained by plastic adherence for 5 days in 48-well plates, were first pretreated or not for 5 min at 37° C. with bpV[pic] (10 μM) or 3TC (0.07 μM). MDM were also pretreated with the indicated concentrations of bpV[pic] (1, 5, and 10 μM) in combination with 0.07 μM 3TC. MDM were subsequently infected with recombinant luciferase-encoding virions (NL4-3 backbone) pseudotyped with macrophage-tropic ADA envelope (10 ng of p24). Infection was allowed to proceed for 48 h and MDM were lysed before monitoring luciferase activity with a microplate luminometer (MLX; Dynex Technologies, Chantilly, Va.). Results shown are the mean±SD for triplicate samples and are representative of two independent experiments.

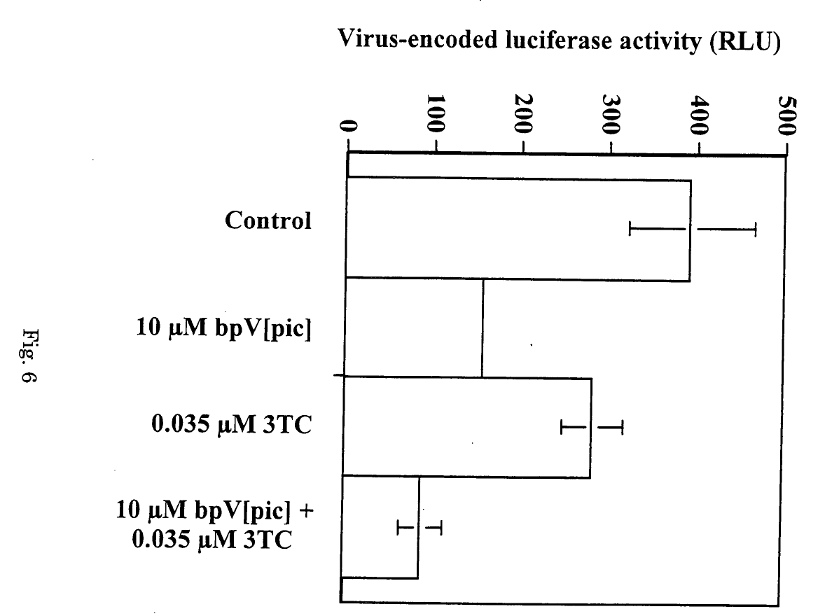

Data from FIG. 7 shows a dose-dependent additive anti-HIV-1 effect following treatment of primary human MDM with 3TC and increasing concentrations of bpV[pic].

### EXAMPLE 8

### Treatment of Human Peripheral Blood Mononuclear Cells With bpV[pic] Leads to an Increase in the Triphosphate Form of 3TC

Human peripheral blood mononuclear cells (PBMC's) were obtained from healthy donors subjected to lymphopheresis for 60 min. The yield of PBMC's ranged from 1.8 to 2.6 billion cells per donor. Twenty-five (25) ml fractions of the cell suspensions (obtained by lymphopheresis) were layered on cushions (15 ml) of Lymphocyte Separation Medium (Wisent) in 50 ml tubes, which were then centrifuged at room temperature at 750×g during 25 min. The purified PBMC's were collected (on top of the Ficoll cushions) and washed 3 times in HBSS (without calcium and magnesium) (500×g, 7 min. at room temperature). The cells were resuspended in RPMI containing 5% FBS at the cell concentration of 25×10⁶ per ml. Incubations were carried out using 1 ml of PBMC's suspension per tube. The cells were next preincubated during 30 min at 37° C. in the presence of 10 μM bpV[pic] or its diluent (HBSS). The cells were then further incubated for 30 min at 37° C. in the presence of 5, 10 or 20 μM 3TC and 1 μCi tritium-labelled 3TC (Moravek) per tube. The incubations were stopped by addition of 2 ml of ice-cold calcium/magnesium-free HBSS and centrifuged at 550×g, 2 min, at 4° C. The supernatants were removed and the pellets were washed twice with calcium/magnesium-free HBSS under the same conditions. The pellets were then denatured by addition of 200 μl of a mixture of acetonitrile and water (50/50, vol/vol), vortexed and let to stand at 0° C. for 60 min. Eight hundred (800) ul of cold (4° C.) water were added and the tubes were then transferred to a hot water bath (95° C.) for 2 min. The samples were then stored at −20° C. until analysis by HPLC. All incubations were performed in triplicates or quadruplicates.

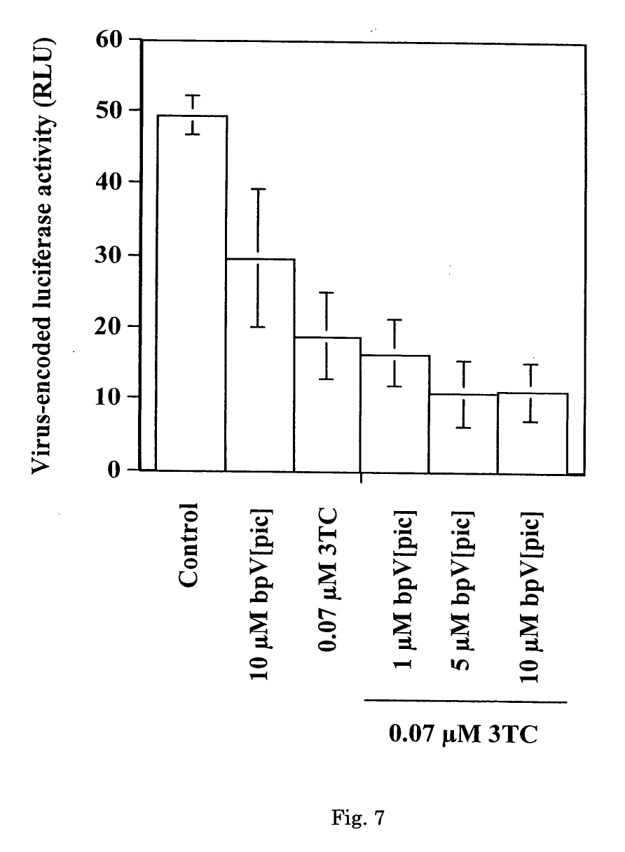

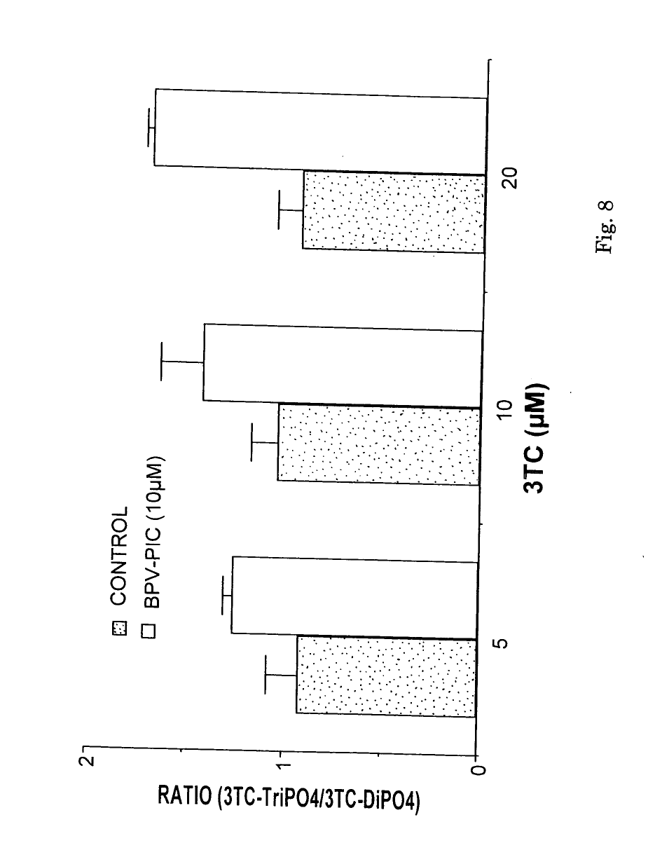

Results from FIG. 8 demonstrate that bpV[pic] at 10 μM consistently increases the ratio of 3TC triphosphate over 3TC diphosphate. This was observed at the three concentrations of 3TC tested (5, 10, and 20 μM). The data shown are the mean of 3 separate experiments; each experiment included triplicate or quadruplicate incubations for each experimental condition tested. In all experiments, and at the three concentrations of 3TC tested, bpV[pic] decreased the formation of 3TC diphosphate and increased the formation of 3TC triphosphate.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for the treatment of an infection in a patient, which comprises administering to said patient a therapeutically effective amount of a bis-peroxovanadium (bpV) compound.

2. The method of claim 1, wherein said bpV compound is a phosphotyrosyl phosphatase inhibitor.

3. The method of claim 2, wherein said bpV compound comprises an oxo ligand, two peroxo anions, and an ancillary ligand located in an inner coordination sphere of vanadate.

4. The method of claim 1, wherein said infection is caused by a virus.

5. The method of claim 1, wherein said patient is a mammal.

6. The method of claim 5, wherein said mammal is selected from the group consisting of human, ovine, bovine, equine, caprine, porcine, feline and canine.

7. The method of claim 2, wherein said patient is a human.

8. The method of claim 7, wherein said virus is a human virus selected from the group consisting of DNA viruses, RNA viruses and Retroviridae.

9. The method of claim 7, wherein said virus is a human immunodeficiency virus.

10. The method of claim 1, wherein the bpV compound is administered intravenously, subcutaneously, intradermally, transdermally, intraperitoneally, orally or topically.

11. The method of claim 1, wherein the bpV compound is administered with a patch or an implant.

12. The method of claim 1, wherein the bpV compound is administered by inhalation.

13. The method of claim 12, wherein the bpV compound is administered with an aerosol spray.

14. The method of claim 12, wherein the bpV compound is in a powder form.

15. The method of claim 1, wherein the bpV compound is in association with a liposomal composition suitable for administration.

16. The method of claim 1, wherein the bpV compound is in a tablet form.

17. The method of claim 1, wherein the bpV compound is administered in combination with an antiviral agent.

18. The method of claim 17, wherein the antiviral agent is selected from the group consisting of nucleoside analogues, protease and neuraminidase inhibitors, interferon a, and non nucleoside analogues.

19. The method of claim 17, wherein the antiviral agent is selected from the group consisting of AZT and 3TC.

20. The method of claim 1, wherein the bpV compound is administered in combination with one or more immunomodulator(s).

21. The method of claim 20, wherein said immunomodulator is selected from the group consisting of leukotrienes, chemokines, cytokines, growth factors and interferons.

22. A method for the enhancement of antimicrobial efficacy of antimicrobial agents, which comprises administering to a patient undergoing an antimicrobial therapy, a therapeutically effective amount of a bis-peroxovanadium (bpV) compound.

23. The method of claim 22, wherein said bpV compound is a phosphotyrosyl phosphatase inhibitor.

24. The method of claim 23, wherein said bpV compound comprises an oxo ligand, two peroxo anions, and an ancillary ligand located in an inner coordination sphere of vanadate.

25. The method of claim 22, wherein said patient is a mammal.

26. The method of claim 25, wherein said mammal is selected from the group consisting of human, ovine, bovine, equine, caprine, porcine, feline and canine.

27. The method of claim 24, wherein said patient is a human.

28. The method of claim 27, wherein said antimicrobial agent is selected from the group consisting of nucleoside analogues, protease and neuraminidase inhibitors, interferon α, and non nucleoside analogues, such as non nucleoside reverse transcriptase inhibitors (NNRTI), chemokines and chemokines antagonists

29. The method of claim 22, wherein the bpV compound is administered intravenously, subcutaneously, intradermally, transdermally, intraperitoneally, orally or topically.

30. The method of claim 22, wherein the bpV compound is administered with a patch or an implant.

31. The method of claim 22, wherein the bpV compound is administered by inhalation.

32. The method of claim 31, wherein the bpV compound is administered with an aerosol spray.

33. The method of claim 32, wherein the bpV compound is in a powder form.

34. The method of claim 22, wherein the bpV compound is in association with a liposomal composition suitable for administration.

35. The method of claim 22, wherein the bpV compound is in a tablet form.

36. A pharmaceutical composition for the treatment of an infection in a patient, which comprises an therapeutically effective amount of a bis-peroxovanadium (bpV) compound in association with a pharmaceutically acceptable carrier.

37. The pharmaceutical composition of claim 36, wherein said bpV compound is a phosphotyrosyl phosphatase inhibitor.

38. The pharmaceutical composition of claim 37, wherein said bpV compound comprises an oxo ligand, two peroxo anions, and an ancillary ligand located in an inner coordination sphere of vanadate.

39. The pharmaceutical composition of claim 36, wherein said infection is caused by a virus.

40. The pharmaceutical composition of claim 36, wherein said patient is a mammal.

41. The pharmaceutical composition of claim 40, wherein said mammal is selected from the group consisting of human, ovine, bovine, equine, caprine, porcine, feline and canine.

42. The pharmaceutical composition of claim 36, wherein said patient is a human.

43. The pharmaceutical composition of claim 42, wherein said said virus is a human virus selected from the group consisting of DNA viruses, RNA Buses and Retroviridae.

44. The pharmaceutical composition of claim 42, wherein said virus is a human immunodeficiency virus.

45. The pharmaceutical composition of claim 36, wherein said pharmaceutically acceptable carrier is adapted to be administered intravenously, subcutaneously, intradermally, transdermally, intraperitoneally, orally or topically.

46. The pharmaceutical composition of claim 36, wherein said pharmaceutically acceptable carrier is adapted to be administered with a patch or an implant.

47. The pharmaceutical composition of claim 36, wherein said pharmaceutically acceptable carrier is adapted to be administered by inhalation.

48. The pharmaceutical composition of claim 47, wherein said pharmaceutically acceptable carrier is adapted to be administered with an aerosol spray.

49. The pharmaceutical composition of claim 48, wherein said pharmaceutically acceptable carrier is in a powder form.

50. The pharmaceutical composition of claim 36, wherein said pharmaceutically acceptable carrier is a liposomal composition.

51. The pharmaceutical composition of claim 36, wherein said composition is in a tablet form.

52. The pharmaceutical composition of claim 36, wherein said composition further comprises an antiviral agent.

53. The pharmaceutical composition of claim 52, wherein the antiviral agent is selected from the group consisting of nucleoside analogues, protease and neuraminidase inhibitors, interferon a, and non nucleoside analogues.

54. The pharmaceutical composition of claim 52, wherein the antiviral agent is selected from the group consisting of AZT and 3TC.

55. The pharmaceutical composition of claim 36, wherein said composition further comprises an immunomodulator.

56. The pharmaceutical composition of claim 55, wherein the immunomodulator is selected from the group consisting of leukotrienes, chemokines, cytokines, growth factors and interferons.
